(19) United States
(12) Patent Application Publication
(10) Pub. No.: US 2025/0197786 A1
(43) Pub. Date: Jun. 19, 2025

(54) CULTIVATION DEVICE

(71) Applicant: HONDA MOTOR CO., LTD., Minato-ku, Tokyo (JP)

(72) Inventors: Minoru Goto, Wako-shi (JP); Shohei Kinoshita, Wako-shi (JP); Nozomi Shiobara, Wako-shi (JP); Kenji Machida, Wako-shi (JP); Mizuho Doi, Wako-shi (JP); Satoshi Shiozaki, Wako-shi (JP)

(21) Appl. No.: 18/845,453

(22) PCT Filed: Feb. 20, 2023
(86) PCT No.: PCT/JP2023/006052

§ 371 Date: Sep. 10, 2024

(30) Foreign Application Priority Data
Mar. 14, 2022 (JP) 2022-039742

(51) Int. Cl. C12M 1/00; C12M 3/00

(52) U.S. Cl. CPC C12M 21/02; C12M 23/14; C12M 23/26; C12M 23/34; C12M 23/48

(57) ABSTRACT

A cultivation device comprises a water retention part and an accommodation part. Retention water is retained in the water retention part. A cultivation liquid and a microalga are accommodated in the accommodation part. The water retention part and the accommodation part are adjacent to one another. At least one section of the accommodation part is in contact with the water retention part. A peripheral section of the accommodation part has a contact region that contacts the retention water when the retention water is retained in the water retention part, and a non-contact region that does not contact the retention water.

### TECHNICAL FIELD

The present invention relates to a culture device (cultivation device) for culturing microalgae.

### BACKGROUND ART

Heretofore, efforts aimed at mitigating climate change or reducing its impact have continued, and toward the realization thereof, research and development in relation to the reduction of carbon dioxide emissions are being carried out. From this point of view, attention has been focused on microalgae. This is because microalgae consume carbon dioxide through photosynthesis. Accordingly, a culture device for culturing microalgae is anticipated as a device that contributes to mitigating climate change or reducing its impact.

In order to cause microalgae to perform photosynthesis, it is necessary to supply light to the microalgae. JP 2013-521783 A describes providing a culturing tank made of a material that transmits light.

### SUMMARY OF THE INVENTION

As microalgae performs photosynthesis more actively, more carbon dioxide is consumed. In order to cause microalgae to perform photosynthesis, it is necessary to supply more light to the microalgae.

The present invention has the object of solving the above-mentioned problem.

According to an embodiment of the present invention, there is provided a culture device comprising: a water storage section configured to store stored water; and an accommodation section configured to accommodate a culture solution and microalgae, wherein the water storage section and the accommodation section are adjacent to each other, at least a part of the accommodation section is in contact with the water storage section, and an outer peripheral portion of the accommodation section includes a contact region that is in contact with the stored water and a non-contact region that is not in contact with the stored water when the stored water is stored in the water storage section.

Light enters the non-contact region without passing through the water storage section (stored water). That is, according to the present invention, it is possible to make light directly enter the non-contact region. In this case, more light reaches the accommodation section than in the case where the entire accommodation section is surrounded by the water storage section. That is, the transmittance of light in the accommodation section is improved. Therefore, the microalgae can actively perform photosynthesis in the entire accommodation section.

As a result, the microalgae sufficiently fix carbon dioxide. Therefore, the culture device enables a sufficient amount of carbon dioxide to be consumed. Thus, the culture device contributes to mitigating climate change or reducing its impact.

### BRIEF DESCRIPTION OF THE DRAWINGS

FIG. 1 is a schematic perspective view of a culture device according to a first embodiment of the present invention;

FIG. 2 is a schematic exploded perspective view of the culture device;

FIG. 3 is a schematic vertical cross-sectional view of the culture device as viewed from a longitudinal direction;

FIG. 4 is a schematic overall perspective view of an upright tank unit forming a water storage section and an accommodation section;

FIG. 5A and FIG. 5B are schematic views showing the process of manufacturing the upright tank unit;

FIG. 6 is a schematic perspective view of an upright guide member;

FIG. 7 is a cross-sectional view taken along line VII-VII in FIG. 6;

FIG. 8 is a front view as viewed from the direction VIII in FIG. 6;

FIG. 9 is a schematic vertical cross-sectional view showing a state where a support body is accommodated in the upright tank unit;

FIG. 10 is a schematic view showing occurrence of convection in the accommodation section;

FIG. 11 is a schematic vertical cross-sectional view of a culture device according to a second embodiment of the present invention, as viewed from the longitudinal direction;

FIG. 12 is a schematic perspective view of an inclined guide member;

FIG. 13 is a cross-sectional view taken along line XIII-XIII in FIG. 12;

FIG. 14 is a front view as viewed from the direction XIV in FIG. 12;

FIG. 15 is a schematic view showing occurrence of convection in the accommodation section; and

FIG. 16 is a cross-sectional view of a body portion of a guide member having a shape different from those in FIGS. 6 and 12.

### DETAILED DESCRIPTION OF THE INVENTION

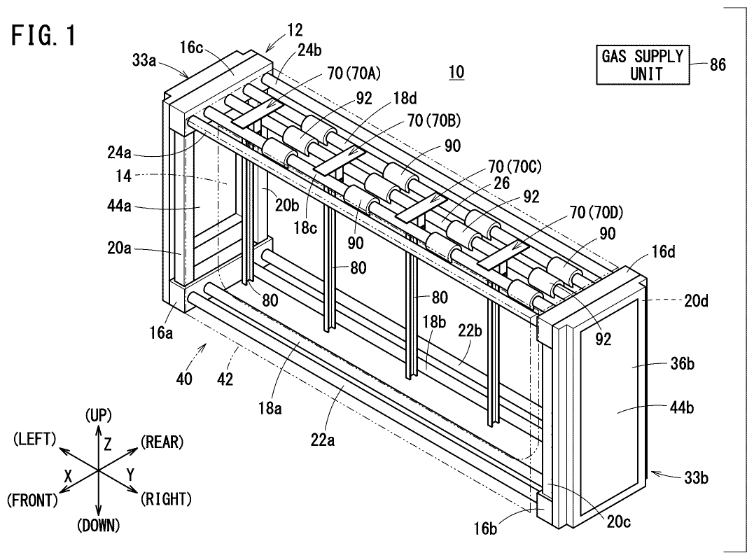

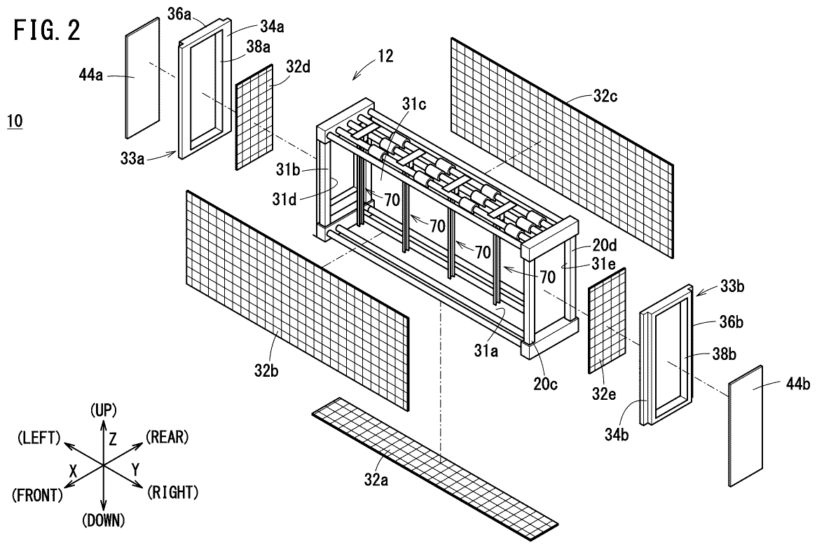

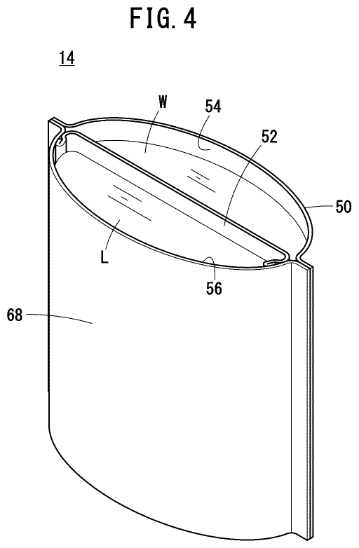

FIG. 1 is a schematic perspective view of a culture device 10 according to a first embodiment. The culture device 10 includes an upright frame 12 serving as a holding member, and an upright tank unit 14 (see FIG. 4) held by the upright frame 12. The upright frame 12 will be described referring to FIGS. 1 and 2. The lower portion of the upright frame 12 includes a first beam portion 16a and a second beam portion 16b, and a first girder portion 18a and a second girder portion 18b extending from the first beam portion 16a toward the second beam portion 16b. The lower ends of a first column portion 20a and a second column portion 20b are joined to the first beam portion 16a. The lower ends of a third column portion 20c and a fourth column portion 20d are joined to the second beam portion 16b. Two reinforcing lower girder portions 22a and 22b are provided between the first beam portion 16a and the second beam portion 16b. The reinforcing lower girder portions 22a and 22b are located outside the first girder portion 18a and the second girder portion 18b (in a direction away from the upright tank unit 14).

The upper portion of the upright frame 12 includes a third beam portion 16c and a fourth beam portion 16d, and a third girder portion 18c and a fourth girder portion 18d extending from the third beam portion 16c toward the fourth beam portion 16d. The upper ends of the first column portion 20a and the second column portion 20b are joined to the third beam portion 16c. The upper ends of the third column portion 20c and the fourth column portion 20d are joined to the fourth beam portion 16d. Two reinforcing upper girder portions 24a and 24b are provided between the third beam portion 16c and the fourth beam portion 16d. The reinforcing upper girder portions 24a and 24b are located outside the third girder portion 18c and the fourth girder portion 18d. The upper portion of the upright frame 12 further includes a support girder portion 26 that extends between the third beam portion 16c and the fourth beam portion 16d. The support girder portion 26 is located between the third girder portion 18c and the fourth girder portion 18d.

In the above description, the first beam portion 16a, the second beam portion 16b, the third beam portion 16c, and the fourth beam portion 16d extend along the arrow X direction in FIGS. 1 and 2. The first girder portion 18a, the second girder portion 18b, the third girder portion 18c, the fourth girder portion 18d, the reinforcing lower girder portions 22a and 22b, the reinforcing upper girder portions 24a and 24b, and the support girder portion 26 extend along the arrow Y direction in FIGS. 1 and 2. The arrow X direction and the arrow Y direction are horizontal directions orthogonal to each other. The first column portion 20a, the second column portion 20b, the third column portion 20c, and the fourth column portion 20d extend along the arrow Z direction in FIGS. 1 and 2. The arrow Z direction is a vertical direction (a gravity direction). The arrow Z direction is orthogonal to the arrow X direction and the arrow Y direction.

As shown in FIG. 2, a mesh 32a is fitted into a first frame portion 31a formed by the first beam portion 16a, the first girder portion 18a, the second girder portion 18b, and the second beam portion 16b. A mesh 32b is fitted into a second frame portion 31b formed by the first girder portion 18a, the first column portion 20a, the third column portion 20c, and the third girder portion 18c. A mesh 32c is fitted into a third frame portion 31c formed by the second girder portion 18b, the second column portion 20b, the fourth column portion 20d, and the fourth girder portion 18d. A mesh 32d is fitted into a fourth frame portion 31d formed by the first beam portion 16a, the first column portion 20a, the second column portion 20b, and the third beam portion 16c. A mesh 32e is fitted into a fifth frame portion 31e formed by the second beam portion 16b, the third column portion 20c, the fourth column portion 20d, and the fourth beam portion 16d. The meshes 32a to 32e improve the strength of the upright frame 12.

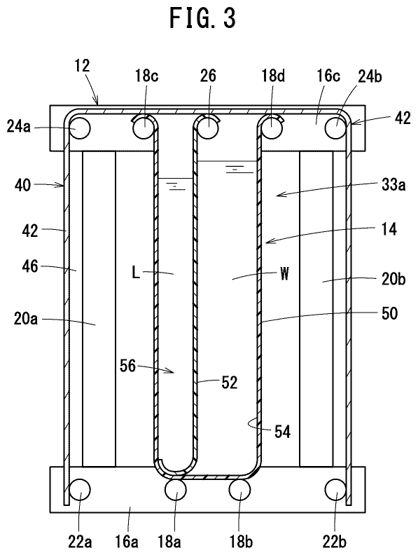

The fourth frame portion 31d and the fifth frame portion 31e having relatively small areas are provided with a first side panel member 33a and a second side panel member 33b, respectively. The first side panel member 33a includes a flange portion 34a and a hollow tubular portion 36a which is bulged in a quadrangular prism shape from the flange portion 34a. The flange portion 34a of the first side panel member 33a is connected to the first beam portion 16a, the first column portion 20a, the second column portion 20b, and the second beam portion 16b via bolts. A side opening 38a is formed in the hollow tubular portion 36a. Similarly, the second side panel member 33b includes a flange portion 34b and a hollow tubular portion 36b which is bulged in a quadrangular prism shape from the flange portion 34b. The flange portion 34b of the second side panel member 33b is connected to the third beam portion 16c, the third column portion 20c, the fourth column portion 20d, and the fourth beam portion 16d via bolts. A side opening 38b is formed in the hollow tubular portion 36b. The first side panel member 33a and the second side panel member 33b are preferably made of a hard material such as stainless steel in order to prevent air from flowing into an air layer 46 (see FIG. 3) from the side portion of the culture device 10. Alternatively, the first side panel member 33a and the second side panel member 33b may be made of a material that transmits light such as a transparent acrylic plate. The air layer 46 will be described later.

The culture device 10 includes a heat insulating unit 40 shown in FIG. 1. In this case, the heat insulating unit 40 includes a main heat insulating sheet 42, a first auxiliary heat insulating sheet 44a, and a second auxiliary heat insulating sheet 44b. The main heat insulating sheet 42, the first auxiliary heat insulating sheet 44a, and the second auxiliary heat insulating sheet 44b are formed of sheet-shaped resin that is flexible and transmits light.

As shown in FIG. 3, the main heat insulating sheet 42 covers the second frame portion 31b, the third frame portion 31c, and an upper opening of the upright frame 12. In this case, the main heat insulating sheet 42 is caught by the reinforcing lower girder portions 22a and 22b, and the reinforcing upper girder portions 24a and 24b. Since the reinforcing lower girder portions 22a and 22b, and the reinforcing upper girder portions 24a and 24b are located outside the upright tank unit 14, clearances are formed between the main heat insulating sheet 42 and the upright tank unit 14. The clearances form the air layer 46 between the main heat insulating sheet 42 and the upright tank unit 14. The air layer 46 thermally insulates the upright tank unit 14 from the atmosphere outside the main heat insulating sheet 42.

Note that, as shown in FIG. 1, the first auxiliary heat insulating sheet 44a covers the side opening 38a of the first side panel member 33a. The second auxiliary heat insulating sheet 44b covers the side opening 38b of the second side panel member 33b.

The upright tank unit 14 will be described. FIG. 4 is a schematic overall perspective view of the upright tank unit 14. The upright tank unit 14 includes a container 50 and a partition portion 52. The partition portion 52 provided in the container 50 divides the interior of the container 50 into a water storage section 54 and an accommodation section 56. The water storage section 54 is a space for storing stored water W, and the accommodation section 56 is a space for accommodating microalgae and a culture solution L. Specific examples of the microalgae include the “Honda DREAMO Strain” deposited in International Patent Organism Depositary, National Institute of Technology and Evaluation. The location of the International Patent Organism Depositary, National Institute of Technology and Evaluation is #120, 2-5-8 Kazusakamatari, Kisarazu-shi, Chiba. The Honda DREAMO strain was deposited on Apr. 22, 2016 under the accession number of FERM BP-22306. In this case, a typical example of the culture solution L is water.

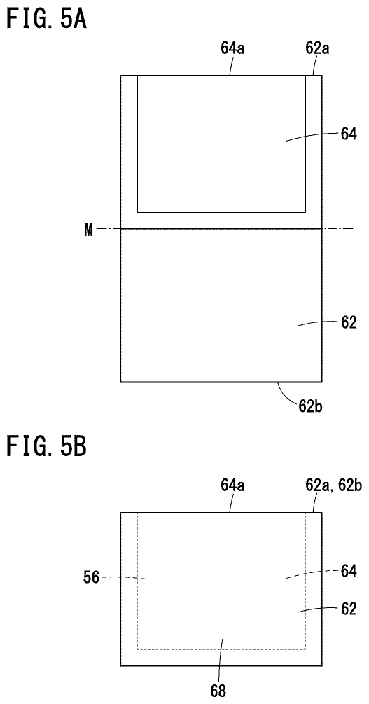

As shown in FIG. 5A and FIG. 5B, the container 50 is formed in a bag shape by a first sheet body 62 being folded. The first sheet body 62 is rectangular. A second sheet body 64 is rectangular or square. The second sheet body 64 is smaller than half the size of the first sheet body 62. The partition portion 52 is formed by joining the second sheet body 64 to the first sheet body 62 before being folded. The first sheet body 62 and the second sheet body 64 are formed of a material that is flexible and transmits light. A typical example of the first sheet body 62 and the second sheet body 64 is linear low-density polyethylene (LLDPE).

Specifically, as shown in FIG. 5A, the second sheet body 64 is placed on the first sheet body 62 before being folded. At this time, an upper side 62a of the first sheet body 62 and an upper side 64a of the second sheet body 64 are aligned. The peripheral edge portions of the second sheet body 64 except the upper edge portion is joined to the first sheet body 62. The upper edge portion of the first sheet body 62 and the upper edge portion of the second sheet body 64 are not joined. The joining is performed by, for example, welding. The same applies to the following.

Next, the first sheet body 62 is folded along a folding line M shown in FIG. 5A, and as shown in FIG. 5B, a lower side 62b of the first sheet body 62 is aligned with the upper side 62a of the first sheet body 62. As a result, the peripheral edge portion of the upper portion of the first sheet body 62 and the peripheral edge portion of the lower portion of the first sheet body 62 overlap each other. Next, among the peripheral edge portions of the first sheet body 62, the overlapping portions of the left and right side portions are joined. In this case as well, the upper edge portions are not joined. As a result, the bag-shaped container 50 whose lower portion and side portion are closed and upper portion is an open end is obtained. The partition portion 52 formed of the second sheet body 64 is provided inside the container 50.

In this manner, the interior of the bag-shaped container 50 is partitioned by the partition portion 52, whereby the water storage section 54 and the accommodation section 56 are formed. The water storage section 54 is a first inner chamber formed by a part of the container 50 and the partition portion 52. The accommodation section 56 is a second inner chamber formed by the remaining part of the container 50 and the partition portion 52. The water storage section 54 and the accommodation section 56 are adjacent to each other. The accommodation section 56 is in contact with the water storage section 54 only via the partition portion 52. That is, in the accommodation section 56, a portion formed by the container 50 is not in contact with the water storage section 54. Hereinafter, this portion is referred to as a non-abutment portion 68. The outer peripheral portion of the accommodation section 56 includes a contact region that is in contact with the stored water W and a non-contact region that is not in contact with the stored water W when the stored water W is stored in the water storage section 54. The contact region and the non-contact region are different locations in the outer peripheral portion of the accommodation section 56. The above-described non-abutment portion 68 is the non-contact region.

The first sheet body 62 (the container 50) and the second sheet body 64 (the partition portion 52) have flexibility, and therefore, in a case where the stored water W is stored in the water storage section 54 and the culture solution L is accommodated in the accommodation section 56, the upright tank unit 14 expands as shown in FIG. 4. At this time, the position of the lower end portion of the water storage section 54 and the position of the lower end portion of the accommodation section 56 substantially coincide with each other. Further, the position of the side end portion of the water storage section 54 and the position of the side end portion of the accommodation section 56 substantially coincide with each other. That is, the size (area) of the second sheet body 64 and the joining region of the first sheet body 62 are designed so that the end portions of the water storage section 54 and the end portions of the accommodation section 56 in the expanded upright tank unit 14 satisfy the above-described relationship.

A plurality of upright guide members 70 (see FIG. 1) are inserted into the accommodation section 56. In FIG. 1, the plurality of upright guide members 70 are arranged at intervals in the width direction of the accommodation section 56 (the arrow Y direction) that lies along the horizontal direction. The distances between respective adjacent upright guide members 70 are substantially equal to each other. Note that, in the first embodiment, a case where the number of the upright guide members 70 is four will be described as an example. However, the number of the upright guide members 70 is not limited to four.

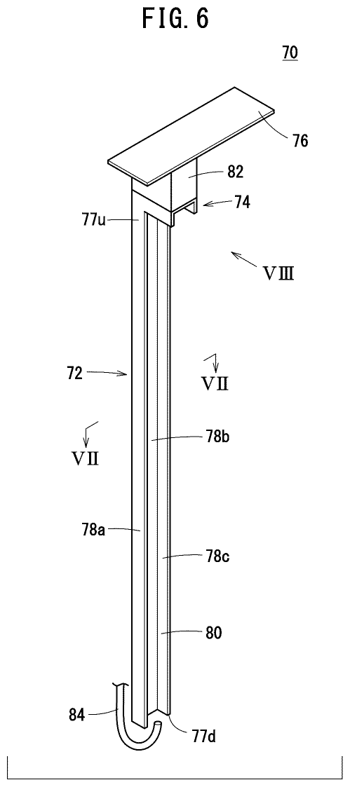

The upright guide members 70 will be described with reference to FIGS. 6 to 8. As shown in FIG. 6, each of the upright guide members 70 includes an upright body portion 72, a horizontal ceiling portion 74, and a plate-shaped supported portion 76.

The upright body portion 72 includes a lower end 77d facing the bottom portion of the accommodation section 56, and an upper end 77u facing the liquid surface of the culture solution L. In this case, the upright body portion 72 extends along the vertical direction (the gravity direction/the arrow Z direction in FIG. 1), similarly to the first column portion 20a to the fourth column portion 20d. Therefore, the lower end 77d of the upright body portion 72 faces downward in the vertical direction, and the upper end 77u of the upright body portion 72 faces upward in the vertical direction.

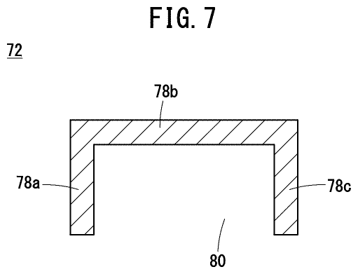

The upright body portion 72 includes a first wall portion 78a, a second wall portion 78b, and a third wall portion 78c. The first wall portion 78a and the third wall portion 78c are separated from each other and face each other with the second wall portion 78b interposed therebetween. In the present embodiment, the first wall portion 78a and the third wall portion 78c are parallel to each other. The first wall portion 78a and the third wall portion 78c may not be parallel to each other. The cross-sectional shape of the upright body portion 72 in a plane perpendicular to the extending direction of the upright body portion 72 is a U-shape as shown in FIG. 7. That is, one side of the upright body portion 72 is open. Hereinafter, the opened one side of the upright body portion 72 is referred to as an intake opening 80.

The upper ends of the first wall portion 78a, the second wall portion 78b, and the third wall portion 78c are bent at approximately 90° with respect to the vertical direction. As a result of this bending, the horizontal ceiling portion 74 is provided in the upright guide member 70. The horizontal ceiling portion 74 is continuous with the upper end 77u of the upright body portion 72.

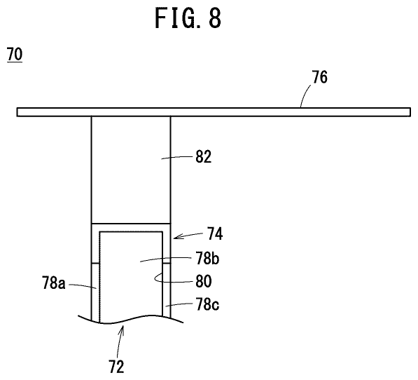

The first wall portion 78a, the second wall portion 78b, and the third wall portion 78c are bent in the above-described direction. As shown in FIG. 8, the cross-sectional shape of the horizontal ceiling portion 74 in a plane perpendicular to the extending direction of the horizontal ceiling portion 74 is a U-shape, similarly to the upright body portion 72. However, the horizontal ceiling portion 74 has an inverted U-shape in which the inner surface (the lower surface) of the bent upper end of the second wall portion 78b serves as the top surface.

As shown in FIG. 6, the plate-shaped supported portion 76 is provided, via a box-shaped spacer 82, on the outer surface (the upper surface) of the upper end of the second wall portion 78b that constitutes the horizontal ceiling portion 74. As shown in FIG. 1, the plate-shaped supported portions 76 are hooked on the third girder portion 18c and the fourth girder portion 18d of the upright frame 12. As a result of this hooking, the upright guide members 70 are supported by the upright frame 12. In FIG. 1, the accommodation section 56 faces the second frame portion 31b, and the water storage section 54 faces the third frame portion 31c. Therefore, in the plate-shaped supported portion 76, the portion extending from the box-shaped spacer 82 toward the third girder portion 18c has a smaller length than the portion extending from the box-shaped spacer 82 toward the fourth girder portion 18d (see FIG. 6).

A gas supply pipe 84 is supported by the upright guide member 70. One end of the gas supply pipe 84 is connected to a gas supply unit 86 shown in FIG. 1. The other end of the gas supply pipe 84 is routed from the back surface of the second wall portion 78b of the upright body portion 72 to the lower end of the intake opening 80. Therefore, the gas supplied from the gas supply unit 86 is discharged from the gas supply pipe 84 at the lower end 77d of the upright body portion 72.

Hereinafter, for convenience, the upright guide member 70 located on the leftmost side in FIG. 1 is referred to as a first guide member 70A. The upright guide member 70 adjacent to the first guide member 70A on the right side is referred to as a second guide member 70B. The upright guide member 70 adjacent to the second guide member 70B on the right side is referred to as a third guide member 70C. The upright guide member 70 located on the rightmost side in FIG. 1 is referred to as a fourth guide member 70D.

When the first guide member 70A to the fourth guide member 70D are inserted into the accommodation section 56, all the intake openings 80 of the first guide member 70A to the fourth guide member 70D face the same direction. Therefore, in FIG. 1, the intake opening 80 of the first guide member 70A faces the second guide member 70B. The intake opening 80 of the second guide member 70B faces the third guide member 70C. The intake opening 80 of the third guide member 70C faces the fourth guide member 70D. The intake opening 80 of the fourth guide member 70D faces the right inner surface of the accommodation section 56. Note that the second wall portion 78b of the first guide member 70A is provided so as to be close to the left side surface of the accommodation section 56.

The horizontal ceiling portion 74 of the first guide member 70A protrudes toward the second guide member 70B. The horizontal ceiling portion 74 of the second guide member 70B protrudes toward the third guide member 70C. The horizontal ceiling portion 74 of the third guide member 70C protrudes toward the fourth guide member 70D. The horizontal ceiling portion 74 of the fourth guide member 70D protrudes toward the right inner surface of the accommodation section 56.

As shown in FIG. 1, the third girder portion 18c and the fourth girder portion 18d are each provided with first clamps 90. The first clamps 90 grip the upper edge portion of the container 50. Consequently, the container 50 is supported by the upright frame 12. The support girder portion 26 is provided with second clamps 92. The second clamps 92 grip the upper edge portion of the partition portion 52. Consequently, the partition portion 52 is supported by the upright frame 12. The first clamps 90 and the second clamps 92 prevent the upright tank unit 14 from sagging when the stored water W is stored in the water storage section 54 and the culture solution L is accommodated in the accommodation section 56.

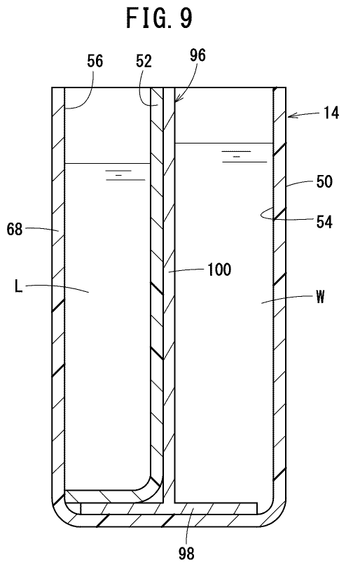

As shown in FIG. 9, a support body 96 may be accommodated in the upright tank unit 14. The support body 96 includes a leg portion 98, and a support wall portion 100 rising substantially perpendicularly from the leg portion 98. The leg portion 98 is located at the bottom portion of the upright tank unit 14 and supports the lower portion of the partition portion 52. The pressure of the stored water W in the water storage section 54 is applied to one surface of the support wall portion 100. This pressure is transmitted to the partition portion 52, whereby the side portion of the water storage section 54 is supported. The pressure of the culture solution L in the accommodation section 56 is applied to the remaining one surface of the support wall portion 100. This pressure is transmitted to the stored water W, whereby the side portion of the accommodation section 56 is supported. The support body 96 is made of a material that transmits light such as a transparent acrylic plate.

The culture device 10 according to the first embodiment is basically configured as described above. Next, the operation and effect of the culture device 10 will be described.

In order to obtain the culture device 10, first, the upright tank unit 14 is manufactured by the above-described procedure. The upright tank unit 14 includes the container 50 formed of the first sheet body 62, and the partition portion 52 formed of the second sheet body 64. In the upright tank unit 14, the water storage section 54 and the accommodation section 56 are formed as two inner chambers by the container 50 and the partition portion 52.

The upright frame 12 is installed outdoors, for example. At this time, the arrow Y direction is preferably set to the east-west direction. This is because in this case, the area of incidence of sunlight on the upright tank unit 14 is increased. Further, the non-abutment portion 68 is directed to the south. As a result, sunlight directly enters the accommodation section 56.

Next, the upright tank unit 14 is held by the upright frame 12. Specifically, the upright tank unit 14 is inserted from between the third girder portion 18c and the fourth girder portion 18d (from the upper opening of the upright frame 12). At this time, the bottom portion of the upright tank unit 14 is directed downward. Thereafter, the upper edge portion of the container 50 is gripped by the first clamps 90 provided on each of the third girder portion 18c and the fourth girder portion 18d. Further, the upper edge portion of the partition portion 52 is gripped by the second clamps 92 provided on the support girder portion 26.

Next, the first guide member 70A is supported by the upright frame 12. Specifically, the upright body portion 72 is inserted into the accommodation section 56 from between the third girder portion 18c and the fourth girder portion 18d (from the upper opening of the upright frame 12) with the upright body portion 72 facing downward and the plate-shaped supported portion 76 facing upward. The dimension of the plate-shaped supported portion 76 along the X direction is larger than the separation distance between the third girder portion 18c and the fourth girder portion 18d. Therefore, one end of the plate-shaped supported portion 76 is hooked on the third girder portion 18c, and the other end of the plate-shaped supported portion 76 is hooked on the fourth girder portion 18d. Consequently, the first guide member 70A is supported by the upright frame 12.

In the same manner, the second guide member 70B to the fourth guide member 70D are supported by the upright frame 12. The separation distances between respective two adjacent upright guide members 70 are made substantially equal to each other. In addition, the intake openings 80 of the first guide member 70A to the fourth guide member 70D are made to face the same direction.

In this state, the stored water W is stored in the water storage section 54. In addition, the culture solution L and microalgae are accommodated in the accommodation section 56. This causes the upright tank unit 14 to expand. At this time, the position of the lower end portion of the water storage section 54 and the position of the lower end portion of the accommodation section 56 substantially coincide with each other. Further, the position of the side end portion of the water storage section 54 and the position of the side end portion of the accommodation section 56 substantially coincide with each other. As described above, the upright tank unit 14 is held by the upright frame 12 by means of the first clamps 90 and the second clamps 92, and therefore the expanded upright tank unit 14 is prevented from sagging.

Next, the main heat insulating sheet 42 is used to cover the second frame portion 31b, the third frame portion 31c, and the upper opening of the upright frame 12. The main heat insulating sheet 42 is caught by the reinforcing lower girder portions 22a and 22b, and the reinforcing upper girder portions 24a and 24b. As a result, as described above, the air layer 46 is formed between the main heat insulating sheet 42 and the upright frame 12. Further, the first auxiliary heat insulating sheet 44a is used to cover the side opening 38a of the first side panel member 33a, and the second auxiliary heat insulating sheet 44b is used to cover the side opening 38b of the second side panel member 33b.

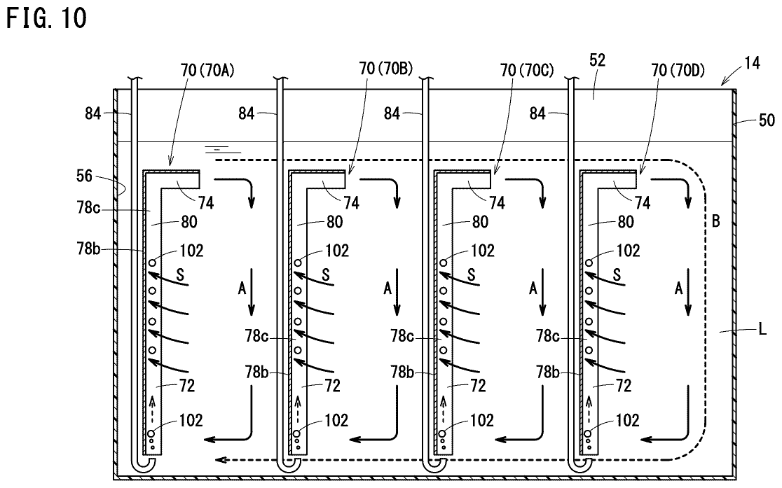

Then, culturing of microalgae is performed. Specifically, gas containing carbon dioxide is supplied from the gas supply unit 86 (see FIG. 1). The gas flows through the gas supply pipes 84 and is discharged from the gas supply pipes 84 at the lower ends 77d of the upright body portions 72 of the upright guide members 70. As schematically shown in FIG. 10, the gas discharged from the gas supply pipes 84 becomes air bubbles 102. The air bubbles 102 rise along the upright body portions 72. In this instance, the intake openings 80 are formed in the upright body portions 72. Therefore, when the air bubbles 102 rise, the culture solution L around the air bubbles 102 is taken in from the intake openings 80 as indicated by the arrow S in FIG. 10. That is, the culture solution L around the air bubbles 102 is entrained in the air bubbles 102. The culture solution L thus entrained rises along the upright body portions 72.

The upright guide members 70 are respectively provided with the horizontal ceiling portions 74. For example, in a case of the leftmost first guide member 70A and the second guide member 70B adjacent to the first guide member 70A, the horizontal ceiling portion 74 of the first guide member 70A protrudes toward the second guide member 70B. Therefore, the culture solution L which is guided by the upright body portion 72 of the first guide member 70A and rises together with the air bubbles 102 advances toward the second guide member 70B. That is, the horizontal ceiling portion 74 changes the direction of movement of the air bubbles 102 and the culture solution L. In this manner, the horizontal ceiling portion 74 is a deflecting portion that directs the air bubbles 102 (gas) and the culture solution L toward one side in the width direction in which the upright guide members 70 are arranged. On one side in the width direction, the adjacent upright guide member 70 or the inner surface of the accommodation section 56 is located.

The air bubbles 102 and the culture solution L moving toward the second guide member 70B come into contact with the second wall portion 78b of the upright body portion 72 of the second guide member 70B. The air bubbles 102 and the culture solution L descend along the second wall portion 78b. At this time, the flow of the culture solution L descending along the upright body portion 72 of the second guide member 70B is prevented from canceling the flow of the culture solution L rising along the upright body portion 72 of the second guide member 70B. The culture solution L that has descended is entrained in the gas discharged from the gas supply pipe 84 in the first guide member 70A as described above. Therefore, the culture solution L that has descended along the upright body portion 72 of the second guide member 70B is drawn to the first guide member 70A side.

The culture solution L drawn to the first guide member 70A side rises by being entrained in the rising air bubbles 102. Therefore, as indicated by the arrows A in FIG. 10, a small convection is generated between the first guide member 70A and the second guide member 70B. Similarly, a small convection is also generated between the second guide member 70B and the third guide member 70C. A small convection is also generated between the third guide member 70C and the fourth guide member 70D. A small convection is also generated between the fourth guide member 70D and the inner surface of the accommodation section 56.

The four small convections are combined, whereby a large convection is generated in the accommodation section 56 as indicated by the arrow B. At this time, since the first guide member 70A to the fourth guide member 70D are respectively provided with the first wall portions 78a and the third wall portions 78c, the air bubbles 102 and the culture solution L are prevented from leaking (escaping) from the side portions of the upright body portions 72. As a result, a large convection can be generated in a favorable manner. The small convections and the large convection cause the air bubbles 102 to spread. That is, carbon dioxide contained in the gas is diffused throughout the culture solution L and sufficiently dissolved in the culture solution L. Further, the microalgae are stirred by the small convections and the large convection. Therefore, the microalgae are prevented from settling or flocculating.

Further, in the first embodiment, the air layer 46 is formed between the main heat insulating sheet 42 and the upright frame 12. The air layer 46 prevents the heat of the atmosphere existing outside the main heat insulating sheet 42 from being transmitted to the accommodation section 56. Further, the accommodation section 56 is cooled by the stored water W stored in the water storage section 54. Therefore, the temperature of the culture solution L is prevented from excessively rising.

In this instance, in the accommodation section 56, the non-abutment portion 68 faces the sun. The non-abutment portion 68 is not in contact with the water storage section 54. Accordingly, the sunlight transmitted through the main heat insulating sheet 42 directly enters the accommodation section 56. Therefore, more sunlight reaches the accommodation section 56 than in the case where the entire accommodation section 56 is surrounded by the water storage section 54. That is, the transmittance of sunlight is improved.

For the reasons described above, microalgae are favorably cultured in the entire accommodation section 56. During culturing thereof, the microalgae sufficiently fix the carbon dioxide by actively carrying out photosynthesis. Consequently, the carbon dioxide is consumed.

As understood from this, in the first embodiment, it is not necessary to provide a stirring blade, a motor, and the like in the culture device 10. Therefore, it is not necessary to excessively increase the rigidity of the upright frame 12. Therefore, it is possible to reduce the size and scale of the culture device 10, and to reduce the investment in equipment. In addition, since microalgae fix carbon dioxide, culturing microalgae can contribute to mitigating climate change or reducing its impact.

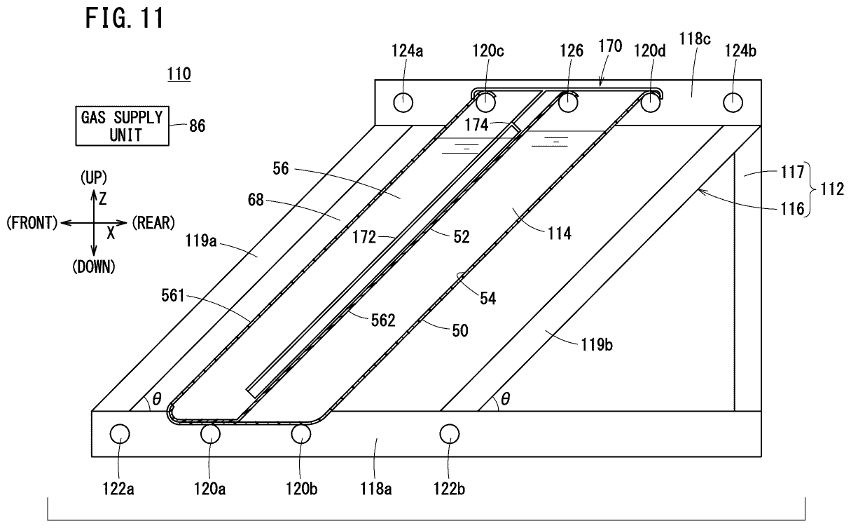

Next, a culture device 110 according to a second embodiment schematically shown in FIG. 11 will be described. Note that the same or corresponding components as those of the culture device 10 are denoted by the same names, and detailed description thereof may be omitted.

The culture device 110 includes an inclined frame 112 serving as a holding member, and an inclined tank unit 114. The inclined frame 112 includes a main frame portion 116, and two back support columns 117. Note that FIG. 11 shows one of the two back support columns 117.

Similarly to the upright frame 12, the main frame portion 116 includes four beam portions, four column portions, four girder portions, four reinforcing girder portions, and one support girder portion 126. FIG. 11 shows two beam portions 118a and 118c among the four beam portions, and two column portions 119a and 119b among the four column portions. The four girder portions are a first girder portion 120a, a second girder portion 120b, a third girder portion 120c, and a fourth girder portion 120d. The four reinforcing girder portions are reinforcing lower girder portions 122a and 122b, and reinforcing upper girder portions 124a and 124b. The reinforcing lower girder portions 122a and 122b are located outside the first girder portion 120a and the second girder portion 120b (in a direction away from the inclined tank unit 114). The reinforcing upper girder portions 124a and 124b are located outside the third girder portion 120c and the fourth girder portion 120d.

The column portion 119a and the column portion 119b are inclined at an angle θ with respect to the beam portion 118a. Similarly, the remaining two columns (not shown) are also inclined at an angle θ with respect to the lower beam portion (not shown). Therefore, the four column portions including the column portions 119a and 119b are inclined at an angle of 90°-θ with respect to the vertical direction. The angle θ is, for example, in a range of 45° to 70°, but is not limited to this range.

The lower end of the back support column 117 is joined to the end portion of the beam portion 118a. The back support column 117 extends along the vertical direction. The upper end of the back support column 117 is joined to one end of the beam portion 118c. The support girder portion 126 is located between the third girder portion 120c and the fourth girder portion 120d.

Although not particularly shown, meshes are fitted into the frame portions of the inclined frame 112, respectively, in the same manner as in first embodiment. Further, the third girder portion 120c and the fourth girder portion 120d are each provided with the first clamps, and the support girder portion 126 is provided with the second clamps (see FIGS. 1 and 2).

The inclined tank unit 114 is provided in accordance with the upright tank unit 14. Specifically, the inclined tank unit 114 includes the container 50 formed of the first sheet body 62, and the partition portion 52 formed of the second sheet body 64. The water storage section 54 and the accommodation section 56 are formed inside the inclined tank unit 114. The inclined tank unit 114 is inclined correspondingly to the main frame portion 116 of the inclined frame 112. Although not particularly shown, the upper end of the inclined tank unit 114 is gripped by, for example, the clamps provided on the third girder portion 120c and the fourth girder portion 120d. Further, in the same manner as in the first embodiment, the heat insulating unit 40 that covers the inclined frame 112 may be provided.

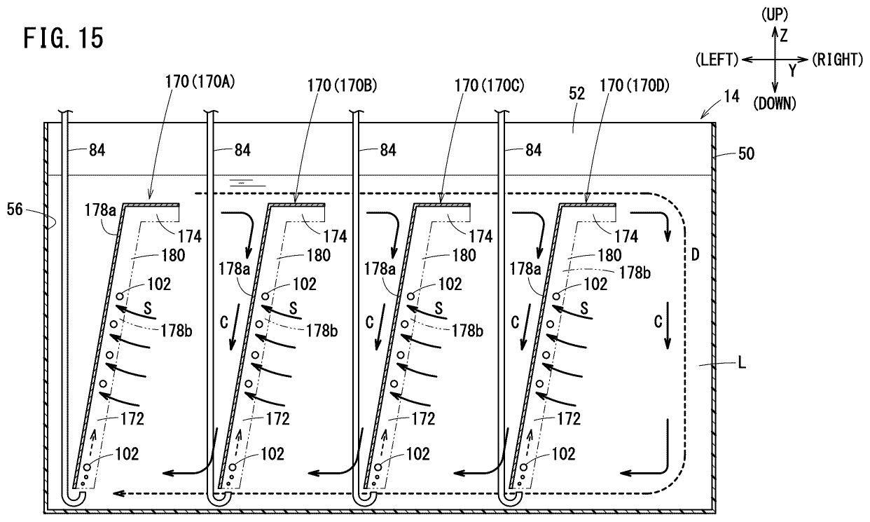

As shown in FIG. 15, a plurality of inclined guide members 170 are inserted into the accommodation section 56. The plurality of inclined guide members 170 are arranged along the arrow Y direction in FIG. 15. The distances between respective adjacent inclined guide members 170 are substantially equal to each other. In the second embodiment, a case where the number of the inclined guide members 170 is four will be described as an example. However, the number of the inclined guide members 170 is not limited to four.

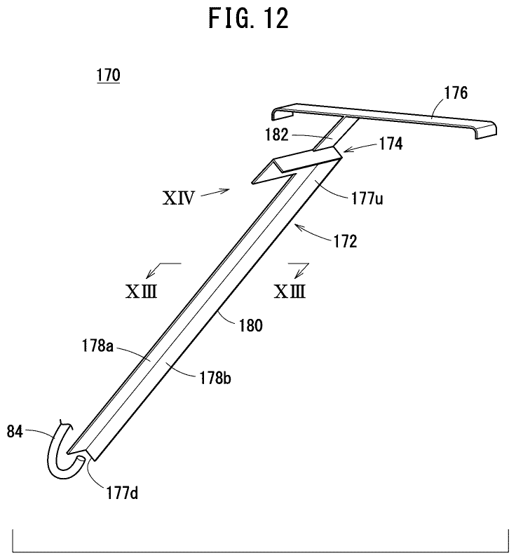

The inclined guide members 170 will be described with reference to FIGS. 12 to 14. As shown in FIG. 12, each of the inclined guide members 170 includes an inclined body portion 172, an inclined ceiling portion 174, and a bar-shaped supported portion 176.

The inclined body portion 172 includes a lower end 177d facing the bottom portion of the accommodation section 56, and an upper end 177u facing the liquid surface of the culture solution L. The inclined body portion 172 is inclined at an angle of 90°-θ with respect to the vertical direction (the gravity direction/the arrow Z direction in FIG. 11), similarly to the four column portions including the column portions 119a and 119b.

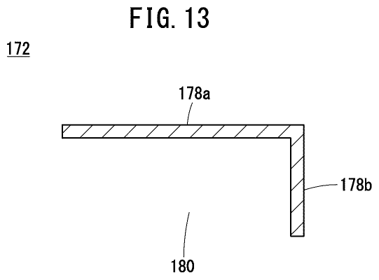

The inclined body portion 172 includes a first wall portion 178a and a second wall portion 178b. The second wall portion 178b is bent so as to intersect with the first wall portion 178a at approximately 90°. Therefore, the cross-sectional shape of the inclined body portion 172 in a plane perpendicular to the extending direction of the inclined body portion 172 is an L-shape as shown in FIG. 13. That is, two sides of the inclined body portion 172 are open, whereby an intake opening 180 is formed in the inclined body portion 172.

When the accommodation section 56 is disposed so as to be inclined, the first wall portion 178a faces, in a substantially parallel manner, a first sidewall 561 of the accommodation section 56 that faces obliquely upward. The upper ends of the first wall portion 178a and the second wall portion 178b are bent at approximately 90° with respect to the vertical direction. As a result of this bending, the inclined ceiling portion 174 is provided in the inclined guide member 170. The inclined ceiling portion 174 is continuous with the upper end 177u of the inclined body portion 172.

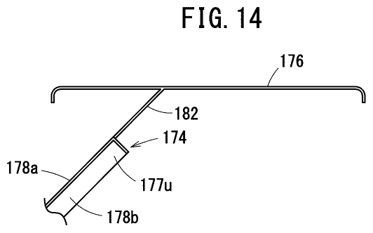

The first wall portion 178a and the second wall portion 178b are bent in the above-described direction, and therefore, when the inclined ceiling portion 174 is viewed from the direction XIV in FIG. 12, the inclined ceiling portion 174 has an L-shape as shown in FIG. 14, similarly to the inclined body portion 172. However, the inclined ceiling portion 174 has an inclined inverted L-shape in which the inner surfaces of the bent upper end portions of the first wall portion 178a and the second wall portion 178b serve as the top surface.

The angle of intersection between the inclined body portion 172 and the inclined ceiling portion 174 is an obtuse angle slightly larger than 90°. The inclined ceiling portion 174 extends along the width direction in which the inclined guide members 170 are arranged. Therefore, when the culture device 110 is viewed from the horizontal direction orthogonal to the width direction in which the inclined guide members 170 are arranged, then as shown in FIG. 15, the inclined guide members 170 are slightly inclined with respect to the vertical direction.

As shown in FIG. 12, the bar-shaped supported portion 176 is provided, via a plate-shaped spacer 182, on the outer surface of the upper end portion of the first wall portion 178a. The plate-shaped spacer 182 is inclined with respect to the vertical direction and the horizontal direction, similarly to the first wall portion 178a. On the other hand, the bar-shaped supported portion 176 extends along the horizontal direction. Therefore, the bar-shaped supported portion 176 is joined to the plate-shaped spacer 182 in a state of being inclined at a predetermined angle with respect to the plate-shaped spacer 182.

The bar-shaped supported portions 176 are hooked on the third girder portion 120c and the fourth girder portion 120d of the inclined frame 112. As a result of this hooking, the inclined guide members 170 are supported by the inclined frame 112. In FIG. 11, the accommodation section 56 faces leftward, and the water storage section 54 faces rightward. Therefore, in the bar-shaped supported portion 176, the portion extending from the plate-shaped spacer 182 toward the third girder portion 120c has a smaller length than the portion extending from the plate-shaped spacer 182 toward the fourth girder portion 120d.

The gas supply pipe 84 is supported by the inclined guide member 170. One end of the gas supply pipe 84 is connected to the gas supply unit 86 shown in FIG. 11. The other end of the gas supply pipe 84 is routed from the back surface of the first wall portion 178a of the inclined body portion 172 to the lower end of the intake opening 180. Therefore, the gas supplied from the gas supply unit 86 is discharged from the gas supply pipe 84 at the lower end of the inclined guide member 170.

Hereinafter, for convenience, the inclined guide member 170 located on the leftmost side in FIG. 15 is referred to as a first guide member 170A. The inclined guide member 170 adjacent to the first guide member 170A on the right side is referred to as a second guide member 170B. The inclined guide member 170 adjacent to the third guide member 170C on the right side is referred to as a third guide member 170C. The inclined guide member 170 located on the rightmost side in FIG. 15 is referred to as a fourth guide member 170D.

When the first guide member 170A to the fourth guide member 170D are inserted into the accommodation section 56, the first wall portions 178a of all of the first guide member 170A to the fourth guide member 170D face vertically upward. In addition, the intake openings 180 of all of the first guide member 170A to the fourth guide member 170D face the same direction. Specifically, in FIG. 15, the intake opening 180 of the first guide member 170A faces the second guide member 170B. The intake opening 180 of the second guide member 170B faces the third guide member 170C. The intake opening 180 of the third guide member 170C faces the fourth guide member 170D located on the rightmost side. The intake opening 180 of the fourth guide member 170D faces the right inner surface of the accommodation section 56. Note that the second wall portion 178b of the first guide member 170A is provided so as to be close to the left side surface of the accommodation section 56.

The inclined ceiling portion 174 of the first guide member 170A protrudes toward the second guide member 170B. The inclined ceiling portion 174 of the second guide member 170B protrudes toward the third guide member 170C. The inclined ceiling portion 174 of the third guide member 170C protrudes toward the fourth guide member 170D. The inclined ceiling portion 174 of the fourth guide member 170D protrudes toward the inner surface of the accommodation section 56.

The culture device 110 according to the second embodiment is manufactured in accordance with the culture device 10, and is installed outdoors, for example. Hereinafter, a case where microalgae are cultured in the culture device 110 will be described.

After the culture device 110 is manufactured, the stored water W is stored in the water storage section 54. In addition, the culture solution L and microalgae are accommodated in the accommodation section 56. This causes the inclined tank unit 114 to expand. At this time, the position of the lower end portion of the water storage section 54 and the position of the lower end portion of the accommodation section 56 substantially coincide with each other. Further, the position of the side end portion of the water storage section 54 and the position of the side end portion of the accommodation section 56 substantially coincide with each other. As in the first embodiment, the inclined tank unit 114 is held by the inclined frame 112 by means of the first clamps 90 and the second clamps 92, and therefore the expanded inclined tank unit 114 is prevented from sagging.

Then, culturing of microalgae is performed. Specifically, gas containing carbon dioxide is supplied from the gas supply unit 86 (see FIG. 11). The gas flows through the gas supply pipes 84 and is discharged from the gas supply pipes 84 at the lower ends of the inclined body portions 172 of the inclined guide members 170. As schematically shown in FIG. 15, the gas discharged from the gas supply pipes 84 becomes air bubbles 102. The air bubbles 102 slightly rise upward in the vertical direction, and then come into contact with the first wall portions 178a of the inclined body portions 172. Thereafter, the air bubbles 102 rise along the first wall portions 178a inclined with respect to the vertical direction. In this manner, according to the second embodiment, even the inclined guide members 170 having the L-shaped inclined body portions 172 can guide the air bubbles 102 in the direction inclined with respect to the vertical direction. That is, in this case, it is possible to cause the air bubbles 102 to advance (rise) according to the inclination of the accommodation section 56.

In this instance, the intake openings 180 are formed in the inclined body portions 172. Therefore, when the air bubbles 102 rise, the culture solution L around the air bubbles 102 is taken in from the intake openings 180, in the same manner as in the first embodiment. That is, the culture solution L around the air bubbles 102 is entrained in the air bubbles 102. The culture solution L thus entrained rises along the inclined body portions 172. The inclined ceiling portions 174 are provided on the upper portions of the inclined guide members 170. The air bubbles 102 stop at the corner where the first wall portion 178a and the second wall portion 178b intersect.

For example, in a case of the leftmost first guide member 170A and the second guide member 170B adjacent to the first guide member 170A, the inclined ceiling portion 174 of the first guide member 170A protrudes toward the second guide member 170B. Therefore, the culture solution L which is guided by the inclined body portion 172 of the first guide member 170A and rises together with the air bubbles 102 advances toward the second guide member 170B. That is, the inclined ceiling portion 174 changes the direction of movement of the air bubbles 102 and the culture solution L. In this manner, the inclined ceiling portion 174 is also a deflecting portion that directs the air bubbles 102 (gas) and the culture solution L toward one side in the width direction in which the inclined guide members 170 are arranged. On one side in the width direction, the adjacent inclined guide member 170 or the inner surface of the accommodation section 56 is located.

Thereafter, four small convections (the arrows C) and one large convection (the arrow D) are generated in the accommodation section 56 in the same manner as in the first embodiment. The small convections and the large convection cause the air bubbles 102 to spread. As a result, carbon dioxide contained in the gas is diffused throughout the culture solution L and sufficiently dissolved in the culture solution L. Further, the microalgae are stirred by the small convections and the large convection. Therefore, the microalgae are prevented from settling or flocculating.

As described above, the angle of intersection between the inclined body portion 172 and the inclined ceiling portion 174 is an obtuse angle of more than 90°. In this case, a larger convection may occur than in the case where the angle of intersection between the inclined body portion 172 and the inclined ceiling portion 174 is an acute angle or a right angle.

For the reasons described above, in the second embodiment as well, microalgae are favorably cultured in the entire accommodation section 56. During culturing thereof, the microalgae sufficiently fix the carbon dioxide by carrying out photosynthesis. Consequently, the carbon dioxide is consumed. That is, the second embodiment can also provide the same effects as those of the first embodiment. Specifically, it is possible to reduce the size and scale of the culture device 110, and to reduce the investment in equipment. In addition, it is possible to contribute to mitigating climate change or reducing its impact.

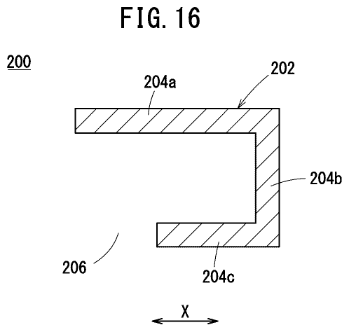

FIG. 16 is a cross-sectional view of a body portion 202 of an inclined guide member 200 in a plane perpendicular to the extending direction of the inclined guide member 200. The body portion 202 of the inclined guide member 200 includes a first wall portion 204a, a second wall portion 204b, and a third wall portion 204c. The first wall portion 204a and the third wall portion 204c are continuous with the second wall portion 204b so as to be orthogonal to the second wall portion 204b. That is, the first wall portion 204a and the third wall portion 204c are substantially parallel to each other. When the accommodation section 56 is disposed so as to be inclined, the first wall portion 204a faces, in a substantially parallel manner, a first sidewall 561 (see FIG. 11) of the accommodation section 56 that faces obliquely upward, and the third wall portion 204c faces, in a substantially parallel manner, a second sidewall 562 (see FIG. 11) of the accommodation section 56 that faces obliquely downward. The protruding length of the first wall portion 204a from the second wall portion 204b is greater than the protruding length of the third wall portion 204c from the second wall portion 204b. The first wall portion 204a is longer than the third wall portion 204c in the front-rear direction of the accommodation section 56 (the arrow X direction). The first wall portion 204a, the second wall portion 204b, and the third wall portion 204c form a U-shape with one short side. An opening formed between the first wall portion 204a and the third wall portion 204c is an intake opening 206.

The inclined guide member 200 is held by the inclined frame 112 in the same manner as the inclined guide member 170, and is inserted into the accommodation section 56. At this time, the first wall portion 204a is directed obliquely upward, and the third wall portion 204c is directed obliquely downward.

In this case, when the gas comes into contact with the third wall portion 204c, the direction of movement of the gas is changed toward the first wall portion 204a. In this manner, the third wall portion 204c narrows the area of the intake opening 206 and functions as a baffle plate. The first wall portion 204a is wider than the third wall portion 204c. Therefore, the gas is prevented from leaking from the intake opening 206.

As described above, the present embodiment discloses the culture device (10, 110) including the water storage section (54) configured to store the stored water (W), and the accommodation section (56) configured to accommodate the culture solution (L) and microalgae, wherein the water storage section and the accommodation section are adjacent to each other, at least a part of the accommodation section is in contact with the water storage section, and the outer peripheral portion of the accommodation section includes the contact region that is in contact with the stored water and the non-contact region (68) that is not in contact with the stored water when the stored water is stored in the water storage section.

Light enters the non-contact region without passing through the water storage section (stored water). That is, according to the present embodiment, it is possible to make light directly enter the non-contact region. In this case, more light reaches the accommodation section than in the case where the entire accommodation section is surrounded by the water storage section. That is, the transmittance of light in the accommodation section is improved.

For this reason, the microalgae actively perform photosynthesis in the entire accommodation section, and sufficiently fix carbon dioxide. Therefore, a sufficient amount of carbon dioxide can be consumed. Thus, the culture device contributes to mitigating climate change or reducing its impact.

The present embodiment discloses the culture device further including the container (50) having flexibility, and the partition portion (52) provided in the container and having flexibility, the water storage section and the accommodation section being formed in the container so as to be adjacent to each other by the partition portion, wherein a part of the container and the partition portion form the water storage section, and a remaining part of the container and the partition portion form the accommodation section.

With such a configuration, the water storage section and the accommodation section that includes the non-contact region can be easily formed.

The present embodiment discloses the culture device wherein the container is formed in a bag shape by folding the first sheet body (62) and joining together the edge portions of the first sheet body, and the partition portion is formed by joining the edge portions of the second sheet body (64) to the first sheet body at locations inward of the edge portions of the first sheet body, and when the water storage section expands by storing the stored water in the water storage section and the accommodation section expands by accommodating the culture solution in the accommodation section, the lower end portion of the water storage section and the lower end portion of the accommodation section are substantially aligned with each other.

According to this feature, the shape of the corner portions can be maintained in the expanded accommodation section and the expanded water storage section. In addition, since the range of the non-contact region is extended, the transmittance of light is further improved.

The present embodiment discloses the culture device wherein when the water storage section expands by storing the stored water in the water storage section and the accommodation section expands by accommodating the culture solution in the accommodation section, the side end portion of the water storage section and the side end portion of the accommodation section are substantially aligned with each other.

In this case, the shape of the corner portions can be further maintained in the expanded accommodation section and the expanded water storage section. In addition, since the range of the non-contact region is further extended, the transmittance of light is yet further improved.

The present embodiment discloses the culture device wherein the culture device includes the support member (26), and the upper portion of the container or the partition portion is supported by the support member.

The present embodiment discloses the culture device wherein the culture device includes the support body (96), and the support body supports the partition portion.

In any case, deformation of the water storage section and the accommodation section is suppressed.

The present embodiment discloses the culture device wherein the culture device includes the heat insulating unit (40) that transmits light and that is configured to cover the water storage section and the accommodation section, and the air layer (46) is formed between the heat insulating unit, and the water storage section and the accommodation section.

The heat insulating unit and the air layer thermally insulate the accommodation section from the outside. Therefore, it is possible to avoid an excessive rise in the temperature of the culture solution.

The present invention is not limited to the above disclosure, and various modifications are possible without departing from the essence and gist of the present invention.

For example, in the first embodiment, similarly to the inclined guide member 170, an upright guide in which the cross section of the body portion has an L-shape may be used. Alternatively, an upright guide in which the cross section of the body portion has a similar shape to that of the inclined guide member 200 may be used. The angle of intersection between the upright body portion 72 and the horizontal ceiling portion 74 may be an obtuse angle, similarly to the angle of intersection between the inclined body portion 172 and the inclined ceiling portion 174 in the second embodiment.

In the second embodiment, similarly to the upright guide member 70, an inclined guide in which the cross section of the body portion has a U-shape may be used.

In the first embodiment and the second embodiment, the light is not limited to sunlight. Alternatively, artificial illumination light can be used.

1. A culture device comprising: a water storage section configured to store stored water; and an accommodation section configured to accommodate a culture solution and microalgae,
wherein the water storage section and the accommodation section are adjacent to each other,
at least a part of the accommodation section is in contact with the water storage section, and
an outer peripheral portion of the accommodation section includes a contact region that is in contact with the stored water and a non-contact region that is not in contact with the stored water when the stored water is stored in the water storage section.

2. The culture device according to claim 1, further comprising a container having flexibility, and a partition portion provided in the container and having flexibility, the water storage section and the accommodation section being formed in the container so as to be adjacent to each other by the partition portion, wherein
a part of the container and the partition portion form the water storage section, and a remaining part of the container and the partition portion form the accommodation section.

3. The culture device according to claim 2, wherein
the container is formed in a bag shape by folding a first sheet body and joining together edge portions of the first sheet body, and the partition portion is formed by joining edge portions of a second sheet body to the first sheet body at locations inward of the edge portions of the first sheet body, and
when the water storage section expands by storing the stored water in the water storage section and the accommodation section expands by accommodating the culture solution in the accommodation section, a lower end portion of the water storage section and a lower end portion of the accommodation section are substantially aligned with each other.

4. The culture device according to claim 3, wherein
when the water storage section expands by storing the stored water in the water storage section and the accommodation section expands by accommodating the culture solution in the accommodation section, a side end portion of the water storage section and a side end portion of the accommodation section are substantially aligned with each other.

5. The culture device according to claim 2, wherein
the culture device comprises a support member, and an upper portion of the container or the partition portion is supported by the support member.

6. The culture device according to claim 2, wherein
the culture device comprises a support body, and the support body supports the partition portion.

7. The culture device according to claim 1, wherein
the culture device comprises a heat insulating unit that transmits light and that is configured to cover the water storage section and the accommodation section, and an air layer is formed between the heat insulating unit, and the water storage section and the accommodation section.
